(19) United States
(12) Patent Application Publication
(10) Pub. No.: US 2009/0306393 A1
(43) Pub. Date: Dec. 10, 2009

(54) PROCESS FOR PREPARING OF, IMPURITIES FREE, SUBSTITUTED 2-BENZIMIDAZOLE-SULFOXIDE COMPOUND

(75) Inventors: Blazenko Bajic, Zagreb (HR); Drazen Cavuzic, Zagred (HR); Nafis Defterdarovic, Zagred (HR); Vesna Miskovic Klecar, Zagreb (HR); Igor Sostaric, Zagreb (HR)

Correspondence Address: FOLEY & LARDNER LLP, 150 EAST GILMAN STREET, P.O. BOX 1497, MADISON, WI, 53701-1497, US

(73) Assignee: PLIVA Hrvatska d.o.o.

(21) Appl. No.: 12/096,268

(22) PCT Filed: Dec. 14, 2006
(86) PCT No.: PCT/GB2006/004667

§ 371 Date: Sep. 23, 2008

(30) Foreign Application Priority Data
Dec. 17, 2005 (GB) 0525710.0

(51) Int. Cl. C07D 401/12

(52) U.S. Cl. 546/273.7

(57) ABSTRACT

The present invention relates to an improved process for the preparation of pantoprazole free from overoxidation impurities. The process enables better control of the process and therefore the quality of the products obtained, avoiding the formation of impurities.

The present invention relates to an improved process for the preparation of 5-(Difluoromethoxy)-2-(((3,4-dimethoxy-2-pyridyl)methyl)sulfinyl)benzimidazole of general Formula (I),

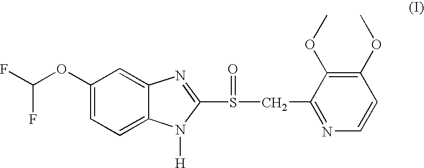

as well as salts thereof. The process enables better control of the process and therefore the quality of the products obtained, avoiding the formation of impurities.

Certain compounds, one of which is also a compound of general Formula (I), or a salt thereof, are used as proton pump inhibitors and are known under their generic names as omeprazole, pantoprazole, rabeprazole, lansoprazole and esomeprazole.

The compounds are widely used for the prevention and treatment of gastric-acid related diseases in mammals and especially in man, including e.g. gastritis, gastric ulcer, duodenitis, duodenal ulcer and gastro-oesophageal reflux.

Processes for their preparation have been disclosed, for instance, in U.S. Pat. No. 4,454,070, U.S. Pat. No. 4,628,098, U.S. Pat. No. 4,758,579, U.S. Pat. No. 5,045,552 and WO2004/111029.

WO2004/111029 describes an oxidation process using chlorine based oxidising agents for the preparation of pantoprazole. The oxidation route is controlled so as to prevent over oxidation by quenching the reaction, such as by the use of sodium metabisulfite. Extensive optimisation work by the application of HPLC is used to discover the appropriate point to add the quenching agent for the specific process parameters used and avoid overoxidation.

A key step in the synthesis of a compound of Formula (I) is the oxidation of the equivalently substituted compound of Formula (II)

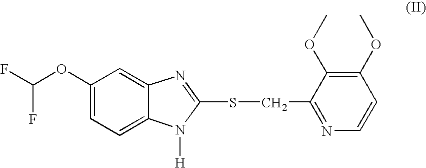

to the corresponding sulfoxide of general Formula (I).

A critical issue is to avoid unwanted further oxidation (overoxidation) of the sulfoxide of the general Formula (I) to the corresponding sulfone of general Formula (III)

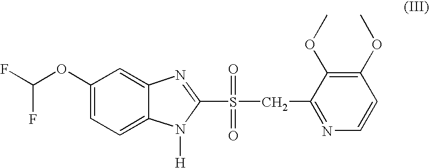

Due to the structural similarity of the sulfoxide (II) and sulfone (III) the removal of the sulfone impurity is very difficult, even the application of high performance chromatography at an industrial scale has been mentioned (U.S. Pat. No. 6,919,459), which is an expensive procedure.

Different approaches, as well as their combinations, have been described to prevent over-oxidation:
application of different, sometimes for industrial use even somewhat extraordinary, oxidizing agents (CA 2 450 433, US 2004/019001)
working with diluted oxidizing agents (WO 2004/063188)
working at low, sometimes even extremely low, temperatures (U.S. Pat. No. 4,758,579, CA 2 450 433)
optimisation of the molar ration of the thioether to oxidizing agent (US 2004/019001).
use of mild oxidising agents such as magnesium monoperoxyphthalate (U.S. Pat. No. 5,391,752)
WO2004111029—optimising several factors in order to achieve a high purity of pantoprazole, including reactor vessel geometry, ratio of reactants, reaction temperature and water content.

All of these oxidation process modifications are in their approach indirect, and mostly result in complicated isolation procedures and reaction conditions that are non-competitive at industrial scale.

Attempts to scale up some of the laboratory procedures described in the literature showed very poor reproducibility and scalability. In particular the reactions are very sensitive even to the small process parameter variations, especially related to reactor geometry and intensity of mixing. Therefore, although the prior art teaches that the reaction can be controlled it can only be controlled by taking extreme care to find the precise reaction conditions and by repeating the process many times to validate the ideal conditions. This makes the reaction extremely inflexible in terms of transferring the process to new facilities or scaling up the production capacity by moving the process to larger reactors.

We have found that even by the use of common oxidizing agents (e.g. sodium hypochlorite) even in high concentrations, that oxidation of thioethers of Formula (II) to sulfoxides of general Formula (I) with a controlled level of thioether impurity of Formula (II) and/or sulfone impurity of Formula (III), is readily achievable giving a reproducible and easily scalable process. The process improvement consists of the control of the molar ratio oxidizing agent vs. thioether (I) by providing an initial inadequate amount of oxidising agent into the process and adding further appropriate amount(s) of oxidising agent according to either of or both of the following two techniques;
1) analysis of the progression of the oxidation reaction at, at least, two time points in the reaction and predicting the amount of further oxidation reagent, by extrapolation, needed to be added in order to complete the reaction and then adding the further amount of oxidation agent sufficient to substantially complete the reaction but insufficient to produce any overoxidation impurities; and/or
2) analysis of the progression of the oxidation reaction by monitoring of the process and adding a further amount of the oxidation reagent and repeating the analysis and addition of further amounts of oxidation agent, if necessary, until the reaction is substantially complete but insufficient to produce any overoxidation impurities.

Therefore, we present as a feature of the invention a process for the preparation of a compound of Formula (I),

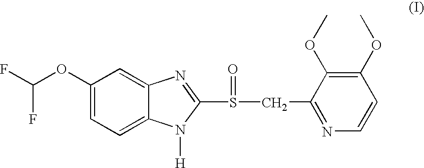

substantially free from impurities (especially overoxidation impurities), the process comprising the following steps;
(1) adding an amount of oxidation reagent to an intermediate of Formula (II),

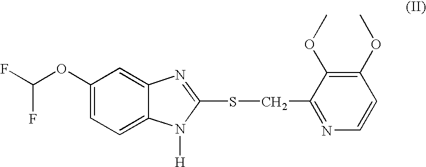

in an amount sufficient to oxidise some of the intermediate of Formula (II), but insufficient to completely oxidise all of the intermediate of Formula (II),
(2) analysing the progression of the oxidation reaction and extrapolating the amount of oxidation reagent needed to be added in order to substantially complete the reaction,
(3) adding the actual amount of oxidation reagent calculated from step (2), and
(4) allowing the reaction to proceed to completion.

By the use of the word “impurity” we mean of any one of the following or a mixture of any thereof; thioether of Formula (II) and/or sulfone impurity of Formula (II), and/or sulfone N-oxide impurity of Formula (IV),

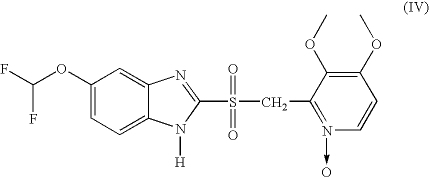

Both the sulfone impurity of Formula (III) and the sulfone N-oxide impurity of Formula (IV) are overoxidation impurities.

By “substantially free” we mean that the process produces not more than 1.0%, 0.8%, 0.9%, 0.8%, 0.6%, 0.5% wt (preferably not more than 0.4, 0.3, 0.2, 0.1, 0.05, 0.04, 0.03, 0.02 and 0.01% wt) of a total amount of impurities, especially overoxidation impurities.

The process produces not more than 0.5% wt (preferably not more than 0.4, 0.3, 0.2, 0.1, 0.05, 0.04, 0.03, 0.02 and 0.01% wt) of a total amount of impurities of Formula (III),

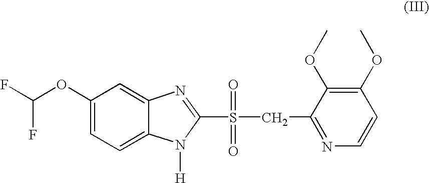

The process produces not more than 0.5% wt (preferably not more than 0.4, 0.3, 0.2, 0.1, 0.05, 0.04, 0.03, 0.02 and 0.01% wt) of an impurity of Formula (II),

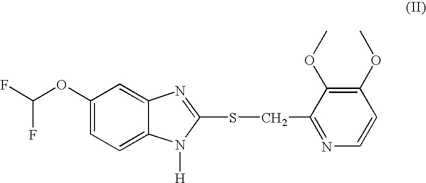

The concentration of impurity (II) is not critical for the final quality of the product since it can be efficiently removed. However, it is preferable that the production of significant amounts is avoided.

The process produces not more than 0.5% wt (preferably not more than 0.4, 0.3, 0.2, 0.1, 0.05, 0.04, 0.03, 0.02 and 0.01% wt) impurity of Formula (IV),

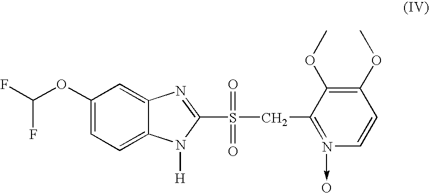

The process produces not more than 0.5% wt (preferably not more than 0.4, 0.3, 0.2, 0.1, 0.05, 0.04, 0.03, 0.02 and 0.01% wt) impurity of Formula (V),

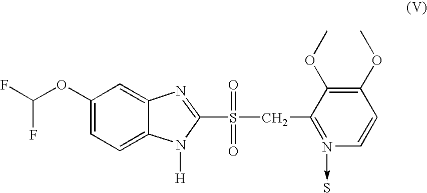

Preferably the compound of Formula (I) is pantoprazole. When the compound of Formula (I) is pantoprazole the compound of Formula (II) is 5-(difluoromethoxy)-2-[[(3,4-dimethoxy-2-pyridinyl)methyl]thio]-1H-benzimidazole, the compound of Formula (III) is 5-(difluoromethoxy)-2-[[(3,4-dimethoxy-2-pyridinyl)methyl]sulfone]-1H-benzimidazole and the compound of Formula (IV) is 5-(difluoromethoxy)-2-[[(3,4-dimethoxy-2-pyridinyl)methyl]sulfone]-1H-benzimidazole-N-oxide.

We present as a feature of the invention a compound of Formula (I) produced by any process of the invention described herein containing less than the amounts of impurity (II) and/or (III) and/or (IV) and/or (V) as defined herein.

In addition we present as a feature of the invention a compound of Formula (I) containing not more than the amounts of impurity (II) and/or (III) and/or (IV) and/or (V) as defined herein.

In addition we present as a feature of the invention pantoprazole produced by any process of the invention described herein containing less than the amount of impurity of 5-(difluoromethoxy)-2-[[(3,4-dimethoxy-2-pyridinyl)methyl]thio]-1H-benzimidazole as defined herein and/or less than the amount of impurity of 5-(difluoromethoxy)-2-[[(3,4-dimethoxy-2-pyridinyl)methyl]sulfone]-1H-benzimidazole and/or 5-(difluoromethoxy)-2-[[(3,4-dimethoxy-2-pyridinyl)methyl]sulfone]-1H-benzimidazole-N-oxide as defined herein.

The exact conditions of the reaction selected are not as critical as those defined in the prior art due to the ability to easily control the reaction according to the techniques described herein. However, generally, the following conditions are preferred.

Preferably the reaction is conducted at a temperature range of below room temperature, preferably less than 0° C. but greater than −10° C.

The reaction can be performed over differing time periods. In practice the period of reaction time is determined by the reactors ability to withdraw released reaction heat at the assigned temperature. With the increasing scale-up reaction time is prolonged (since specific heat exchange surface is decreasing). Reaction times have been in the range of 1 to 5 hours. Depending on the reactor size it could be over 10 hours for large scale reactors suitable for production.

A preferred organic solvent is butyl acetate, ethyl acetate, isobutyl acetate, methyl acetate, dichloromethane, dioxane, acetonitrile. A preferred solvent is ethyl acetate.

The oxidation agent used in the process can be any of the typical oxidising agents used in reaction of the type described, such as sodium bromite, benzoyl peroxide, 2-nitrobenzenesulfininyl chloride/potassium superoxide, N-sulfonyloxaziridines, hypochlorite, cerium ammonium nitrate, tert-butylhydroperoxide, dimethyl dioxirane, perborate, periodate, acyl nitrates, ruthenium tetroxide, peroxy monosulfate, ozone, oxygen, manganese(III) acetylacetonate, iodosylbenzene, 2-hydroperoxyhexafluoro-2-propanol, 1,3-dibromo-5,5-dimethylhydantoin, N-chloro or N-bromo succinimide, permanganates, hydrogen peroxide, m-chloroperoxybenzoic acid, monoperoxyphtalate. Preferably the oxidising agent is sodium hypochlorite, which is cheap and environmentally friendly. Preferably the oxidation agent is added as an aqueous solution.

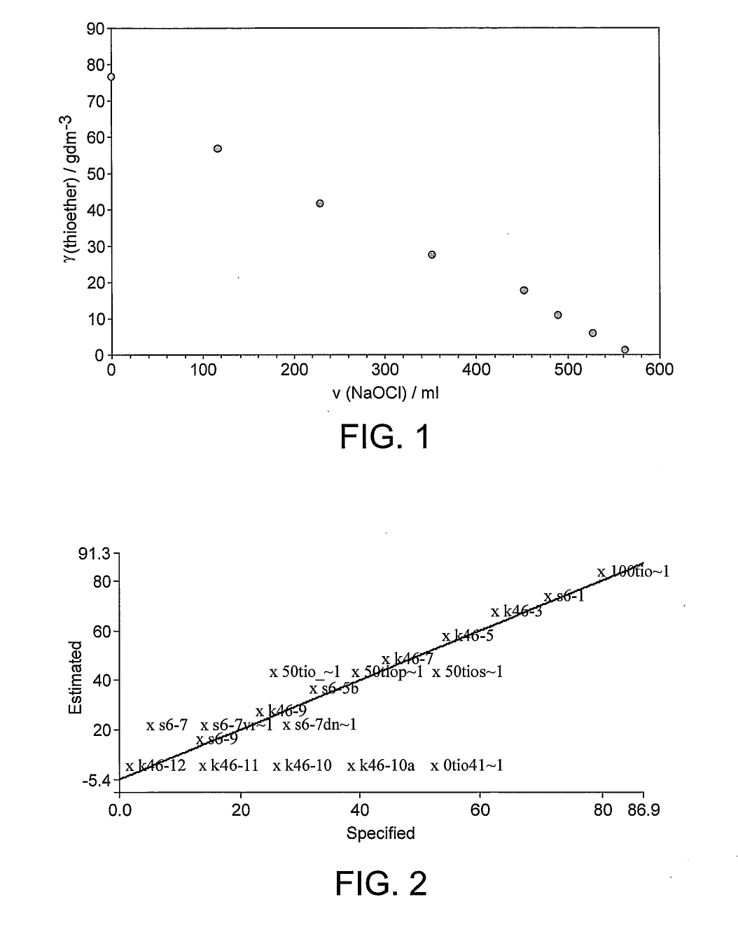

By the use of the term “extrapolate” we mean that; 1) the reaction is analysed; 2) the reaction progress is established according to a calibration produced prior to the reaction and; 3) a prediction is made as to the amount of oxidation reagent that needs to be added to the reaction, sufficient to substantially complete the reaction but insufficient to produce any overoxidation impurities. We have found that the reaction progress shows a near linear dependence to the amount of oxidizing agent added. This fact enables extrapolation of the oxidant quantity necessary to achieve the product quality during the reaction. The reaction progress curve is shown in FIG. 1.

As described above, and as a preferred feature of the invention the applicant has found that by previously running the reaction and analysing the progression of the oxidation reaction a “calibration” of the specific reactor, conditions, reagents and other factors used in the reaction can be achieved such that a extrapolation can be made of the amount of oxidation agent that is necessary to be added to the reaction. This avoids the need to continuously monitor the progression of the oxidation reaction.

By “analysis” we preferably mean the real time analysis of the oxidation reaction. The skilled person will appreciate that such a term as “real time analysis” defines a term of art to which some time delay is experienced between the sampling of the reaction mixture and the result of the analysis. By the real time analysis we mean that the time period between the sampling and the result of analysis is less than 10 minutes, ideally less than 10, 9, 8, 7, 6, 5, 4, 3, 2, 1 minute, 30, 20, 10, 5, 4, 3, 2, 1 seconds. Preferably to reduce the amount of time in a real time analysis system it is better to provide an in-situ analysis. By “in-situ” we mean analysis is performed on the progression of the reaction directly in the reaction vessel. Typically any standard technique in quantitative analysis can be used such as mid IR, near IR, far IR, Fourier transform infrared spectroscopy (FT-IR), Raman, or other infrared spectroscopic measuring signals. Moreover, non IR spectroscopic techniques can also be used including NMR, electronic paramagnetic resonance (EPR), mass spectroscopy, circular dichroism (CD), and other spectroscopic methods which rely on detection of signals outside the IR range. Preferred quantitative analysis techniques include IR and FT-IR techniques.

Depending upon the analysis technique chosen then the skilled person will appreciate that a sample of the reaction may need to be taken in order to determine the progression of the oxidation reaction. Since the reaction is typically performed in a two phase aqueous/organic conditions a small amount of time is needed in order to allow the separation of the two phases and to analyse the aqueous and/or organic phase, ideally less than 10, 9, 8, 7, 6, 5, 4, 3, 2, 1 minute.

Analytical techniques that require the taking of a sample include all above mentioned techniques. Analytical techniques that enable measurements to be made of the reaction progression without the need to take a sample, i.e. in situ analysis include the use of Near and Mid IR and Raman spectroscopy via the use of fibre optics and probes.

As a preferred feature of the invention we provide real-time, in situ analysis.

By using the term “the progression of the oxidation reaction” we mean that either the formation of the compound of Formula (II) is analysed or the consumption of the compound of Formula (I) is analysed, or both.

In-situ analysis of the progress of the reaction is an alternative embodiment. This embodiment, in addition, allows quick real-time analysis and automated process control.

We further present a process for controlling a manufacturing process as defined herein wherein the process is controlled by the use of infra-red detection in situ and in real-time of the compound of Formula (I), or of the compound of Formula (II), during the manufacturing process, analysing the infra-red spectra to determine the amount of compound of Formula (I), or of the compound of Formula (II), present in the reaction mixture; and generating at least one control signal in response to the amount of compound of Formula (I), or of the compound of Formula (II), present in the reaction mixture, wherein the at least one control signal directly or indirectly enables the amount of oxidation agent to be added to the reaction.

Suitable intermediates for compounds of Formula (I) are listed above. Purity levels of the compound of Formula (I) achieved by the use of such a process are as described above.

Suitable in-situ infra-red systems are known as is the software for analysis and control of the process. For example, FTIR systems can be used such as the ReactIR® reaction analysis system sold by Applied Systems, Inc. a corporation in Maryland. See, User's Guide, React IR® and React IR MP® Mobile Reaction Analysis Systems, 3^{rd} Ed., ASI, Applied Systems, Millersville, Md. 1997 (incorporated in its entirety herein by reference). Other systems include, for example, an FTS-6000 system, available from Bio-Rad Laboratories, Hercules, Calif., a Chem-Eye System available from Orbital Sciences, Corp., Dulles, Va., a Foss IR/NIR system, available from Foss North America, Inc., Eden Prairie, Minn., and a Magna-IR 550 Spectrophotometer available from Nicolet Instrument Corp., Madison, Wis. These examples of FTIR systems are illustrative and are not intended to limit the present invention. Other FTIR systems can be used as would be apparent to a person skilled in the art given the description herein.

Generally such systems use a source that emits an infrared beam through optics and sensor to the molecules in a process stage. An optics control module controls components and optics. For example, the optics control module can control focusing, shutters, and any other optical mechanical function. A CPU coordinates with the electronics control module and the optics control module to control irradiation of the molecules being monitored in the reaction.

Chemical bonds absorb infrared energy at specific frequencies (or wavelengths). The structure of compounds can be determined by the spectral locations of infrared absorption. The plot of a compound's infrared transmission versus frequency is called an IR spectrum. This IR spectrum when compared to a reference IR spectrum identifies the molecule. Radiation transmitted from sensor through the material is detected at an infra-red detector. Such an infra-red detector can include but is not limited to a mercury cadmium telluride (MCT) detector or a dueturated triglycine sulfate (DTGS) detector. The IR detector transduces the infrared beam which passed through, or reflected from, the material being analysed into an electrical signal which is provided to the CPU. The CPU mathematically transforms the detected signal representing an interferogram into the wave number domain in the form of a single beam spectrum.

A user can provide further control of the process through graphical user interface from the CPU. In addition to conventional process controls, additional controls for setting and performing FTIR measurements can be provided by the graphical user interface. For example, buttons, sliders, dial wheels, text fields, pull down menus, or other inputs can be provided at graphical user interface to control the process in response to the FTIR measurements.

In the process step, calibration of FTIR system is performed. Calibration involves, among other things, generating reference infra-red spectra at various concentrations of the sample used in the process. A calibration curve is generated.

An infra-red spectra of the background (e.g., air) is measured (also called taken). An infra-red spectra of the solvent is taken. Note an infra-red spectra of the solvent may be previously stored. The background spectra taken is subtracted from the solvent spectra taken to obtain a first difference output representing the solvent spectra only.

An infra-red spectra of a sample is also taken, based on the input received from the FTIR sensor (that is, the electric signal(s) output from IR detector). The initial measured sample spectra, however, includes solvent and background spectra information. The background spectra taken is subtracted from the sample spectra taken to obtain a second difference output. The first and second difference outputs are then subtracted to obtain an infra-red spectra of the reaction mixture.

This reaction mixture IR spectra is compared to a calibration curve to obtain the amount of compound of Formula (I) and/or (II) present in the reaction mixture.

Any conventional FTIR routine that correlates the intensity of a detected infra-red spectra with concentration can be used. Such a routine can be based on a relationship such as Bier's law and/or calibration data relating to the various spectra at known concentrations.

The amount of the molecule [xi] determined is then fed to a process model. The process model determines an [xi,R] representing what the amount of molecule should be at the current time (that is, at the time in which the FTIR measurement and control is being made), and a gamma ([gamma]) value representing what degree of tolerance is allowed before feedback control is undertaken.

A difference ([delta]) between the amount of the molecule [xi] determined and the [xi,R] value output is obtained. The difference delta ([delta]xi) is compared to [gamma]. If the difference [delta]xi is less than [gamma] (indicating the amount of molecule is within an acceptable tolerance), the routine ends (that is, no feedback control action is taken at this iteration. On the other hand, if the difference [delta]xi is equal to or greater than the gamma (indicating the amount of molecule is not within an acceptable tolerance) then data representing the amount of control to be applied is generated. In one example, the difference [delta]xi and [gamma] values are input to a function to determine the amount of control data. In this way, the degree of the control response for the reaction stage can be based upon the value of the [gamma] for a particular process model and the amount of the difference [delta] xi.

The control response can either be displayed to the user who may then control the process, for example by the addition of a further amount of oxidation agent into the process, or it may directly control an actuator that is linked to directly control the process, for example an actuator linked to a hopper that can release further amounts of oxidation agent into the process.

FIG. 1 discloses the linearity of the formation of (I).

FIG. 2 shows calibration curve obtained by Partial Least Squares (PLS) calibration model

### EXAMPLES

Process for better control of impurity level is supported by the IR spectroscopy method for the reaction progress monitoring. For that purpose a multivariate Partial Least Squares (PLS) calibration model was developed. Calibration diagram is shown in FIG. 2. and calibration statistics in Table 2.

### Brief Description of the Method:

Aliquots of the reaction mixture were taken into appropriate vials and left standing still for approximate 2 minutes for the layers to separate. IR spectra of the upper, organic layer was taken by means of the FT-IR spectrometer using liquid cell with CaF₂ windows and pathlength of 0.05 mm. Spectra were recorded in the range 1900-900 cm⁻¹ with 4 cm⁻¹ resolution and 16 consecutive scans. The same cell filled with wet ethyl acetate was used for background spectrum.

Recorded spectra were then analysed using previously developed multivariate PLS calibration model in order to calculate composition of the reaction mixture, specifically mass concentration of thioether. As a standards for calibration both solutions of thioether (II) of known concentrations and reaction mixtures from laboratory experiments with thioether (II) concentrations determined by HPLC were used.

### Brief Summary of Calibration Model:

| | |
|---|---|
| Method Name: | Tioeter 2 |
| Ident: | Spectrum QUANT + v4.51 |
| Description: | Tioeter - nova metoda |
| No. of properties: | 1 |
| No. of standards: | 19, in the concentration range 0 to 79 g/l |

### Calculation Parameters:

| Algorithm: | PLS1 |
|---|---|
| Range: | 1882 to 900 cm⁻¹ |
| Blank regions: | 1762 to 1713 cm⁻¹, 1384 to 1366 cm⁻¹, |
| | 1294 to 1212 cm⁻¹, 1066 to 1031 cm⁻¹ |
| Interval: | 2 cm-1 |
| Analysis Type: | Absorbance |
| Scaling (Spectra): | Mean |
| Scaling (Property): | Mean |
| Smooth: | None |
| Baseline correction: | Derivative |
| Order: | 1 |
| Width: | 5 |
| Normalization: | None |
| Number of factors: | 6 |

TABLE 2
Statistics of calibration model

| Property | Thioether (II) |
|---|---|
| Std Error of Prediction | 1.262 |
| Multiple Correlation | 0.9995 |
| Mean Property Value | 28.47 |
| % Variance (R squared) | 99.91 |
| Std Error of Estimate (SEE) | 0.9305 |
| F-value | 2181 |

The concentration of the thioether (II), sulfone (III) and 5-(difluoromethoxy)-2-[[(3,4-dimethoxy-2-pyridinyl)methyl]sulfone]-1H-benzimidazole-N-oxide (IV) impurities in the product was determined by reversed phase high performance liquid chromatography with UV detection.

### Examples 1-4

### 5-(Difluoromethoxy)-2-[[(3,4-dimethoxy-2-pyridinyl)methyl]sulfinyl]-1H-benzimidazole sodium salt monohydrate

### (Pantoprazole Sodium Salt Monohydrate)

Examples 1-4 were performed in laboratory glass reactor equipment.

### General Procedure:

A glass reactor equipped with a stirrer is charged with ethyl acetate (3286 mL) and 5-(difluoromethoxy)-2-[[(3,4-dimethoxy-2-pyridinyl)methyl]thio]-1H-benzimidazole (“thioether”) (260.0 g) is added. The flask is cooled to −5° C., and mixture of 275.6 mL aqueous 16.9% active sodium hypochlorite solution (or corresponding quantity depending on active chlorine assay) and 10% sodium hydroxide solution (291.2 g) is dropped in approximately 1 hour. Oxidation reaction is monitored by IR spectroscopy method as described above. After the aimed chemical conversion is achieved by the addition of extra oxidant defined through extrapolation, as described above, the reaction is stopped by interrupting the addition of further amounts of sodium hypochlorite. After stirring for a 5 minutes, aqueous 10% Na₂S₂O₃ (614 g) is added dropwise over 15 minutes, additionally stirred for 15 minutes, the phases are then separated. The organic layer is washed twice with 15% sodium carbonate solution (1390 mL). To the organic layer activated charcoal (13 g) is added and after stirring for 15 minutes mixture is filtrated. Organic layer is concentrated to give residue (600 mL) and after cooling to the room temperature acetone (858 mL) is added. By cooling the solution to 5° C. and stirring it for 2 hours at this temperature crystallization occurs. A precipitate is filtered off to give pantoprazole sodium salt monohydrate.

| | | | | | | | | Content |
|---|---|---|---|---|---|---|---|---|
| | | | | | | | | of |
| | | | | | Yield of | Content | Content | Sulfone |
| | | | | Molar Ratio | Pantoprazole | of | of | N- |
| | | Revolution | Reaction | Thioether | Sodium Salt | Sulfone | Thioether | oxide |
| | Agitator | speed | Conversion | vs. Sodium | Monohydrate | (III) | (II) | (IV) |
| Ex | Type | [rpm] | [%] | Hypochlorite | [%] | [%] | [%] | [%] |
| 1. | Impeller | ~250 | 94 | 1:1.82 | 67.9 | 0.03 | 0.19 | 0.05 |
| 2. | Impeller | ~250 | 93 | 1:1.82 | 68.1 | 0.03 | 0.06 | 0.01 |
| 3. | Anchor | ~190 | 95 | 1:1.79 | 65.0 | 0.03 | 0.16 | 0.01 |
| 4. | Anchor | ~170 | 98 | 1:1.92 | 71.5 | 0.038 | 0.14 | 0.01 |

### 5-(Difluoromethoxy)-2-[[(3,4-dimethoxy-2-pyridinyl)methyl]sulfinyl]-1H-benzimidazole sodium salt monohydrate

### (Pantoprazole Sodium Salt Monohydrate)

The above results show that the reaction conditions can be varied to a large degree but using the process of the invention high quality pantoprazole is produced with low amounts of sulfone (III) present.

Examples 5 and 6 were performed in pilot scale (50 litre reactor)

### General Procedure

Into a reactor with impeller agitator is charged ethyl acetate (13.9 L) and after that 5-(difluoromethoxy)-2-[[(3,4-dimethoxy-2-pyridinyl)methyl]thio]-1H-benzimidazole (“thioether”) (1.1 kg) is added. The reactor is cooled to

−5° C., and mixture of 3.75 kg aqueous 15.2% active sodium hypochlorite solution (or corresponding quantity depending on active chlorine assay) and 10% sodium hydroxide solution (2.91 kg) is dropped for 90 minutes. The oxidation reaction is monitored by IR spectroscopy method. After the chemical conversion is achieved, reaction is stopped by stopping of the addition of further amounts of sodium hypochlorite. After stirring for a 15 minutes, aqueous 10% Na₂S₂O₃ (2.6 kg) is added drop wise with aim of the decomposition of any possible unreacted sodium hypochlorite. The content of reactor is stirred for an additional 15 minutes and the aqueous layer is separated from organic layer. The organic layer is washed twice with 15% sodium carbonate solution (11.7 L). To the organic layer activated charcoal (60 g) in ethyl acetate (0.5 L) is added and after stirring for 15 minutes mixture is filtrated. Organic layer is concentrated to give residue (2.5 L) and after cooling to the room temperature acetone (3.6 L) is added. By cooling the solution to 5° C. and stirring it for 2.5 hours at this temperature crystallization occurs. A precipitate is filtered off to give pantoprazole sodium salt monohydrate.

| | | | Molar | Yield of | | | Content |
|---|---|---|---|---|---|---|---|
| | | | Ratio | Pantoprazole | Content | Content | of |
| | | | Thioether | Sodium | of | of | Sulfone |
| | Revolution | Reaction | vs. | Salt | Sulfone | Thioether | N- |
| | speed | Conversion | Sodium | Monohydrate | (III) | (II) | oxide(IV) |
| Example | [rpm] | [%] | Hypochlorite | [%] | [%] | [%] | [%] |
| 1. | 150 | 93 | 1:1.82 | 72 | 0.020 | 0.19 | 0.01 |
| 2. | 150 | 98 | 1:2.02 | 74 | 0.022 | 0.26 | 0.00 |

Having thus described the invention with respect to certain preferred embodiments and further illustrated it with examples, those skilled in the art may come to appreciate substitutions and equivalents that albeit not expressly described are taught and inspired by this invention.

1. A process for the preparation of a compound of Formula (I), 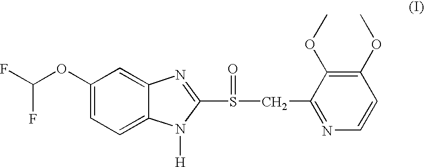
substantially free from impurities, the process comprising the following steps:
(1) adding an amount of oxidation reagent to an intermediate of Formula (II), 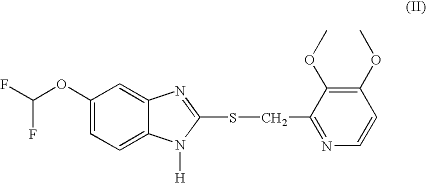
in an amount sufficient to oxidise some of the intermediate of Formula (II), but insufficient to completely oxidise all of the intermediate of Formula (II),
(2) analysing the progression of the oxidation reaction and extrapolating the amount of oxidation reagent needed to be added in order to substantially complete the reaction,
(3) adding the actual amount of oxidation reagent calculated from step (2), and
(4) allowing the reaction to proceed to completion.

2. A process as claimed in claim 1 wherein, either the formation of the compound of Formula (I) is measured or the consumption of the compound of Formula (II) is measured.

3. A process as claimed in claim 1, wherein the process produces not more than 0.5% wt of an impurity of Formula (III)

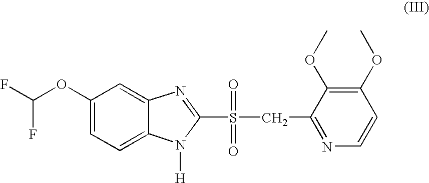

4. A process as claimed in claim 1, wherein the process produces not more than 0.5% wt of an impurity of Formula (II)

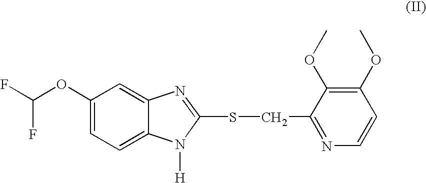

5. A process as claimed in claim 1, wherein the process produces not more than 0.5% wt of an impurity of Formula (IV),

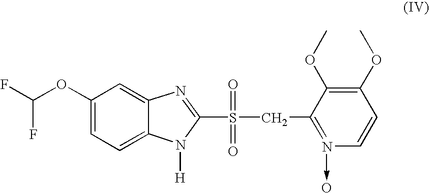

6. A process as claimed in claim 1, wherein the process produces not more than 0.5% wt of an impurity of Formula (V),

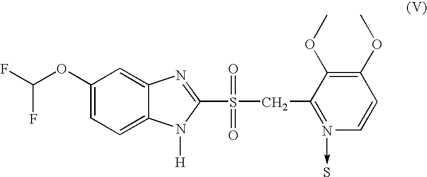

7. A process as claimed in claim 1, wherein the process is controlled by the use of infra-red detection in situ and in real-time of the compound of Formula (I), or of the compound of Formula (II), during the manufacturing process, analysing the infra-red spectra to determine the amount of compound of Formula (I), or of the compound of Formula (II), present in the reaction mixture; and generating at least one control signal in response to the amount of compound of Formula (I), or of the compound of Formula (II), present in the reaction mixture, wherein the at least one control signal directly or indirectly enables the amount of oxidation agent to be added to the reaction.

8. A compound of Formula (I) produced by the process as claimed in claim 1 containing not more than 0.5% wt of any overoxidation impurity.

9. A compound of Formula (I) produced by any process as claimed in claim 1 containing not more than 0.5% wt of a compound of Formula (II) and/or not more than 0.5% wt of a compound of Formula (III).

10. A compound of Formula (I) containing not more than 0.5% wt of a compound of Formula (II) and/or not more than 0.5% wt of a compound of Formula (III).
